(19) United States
(12) Patent Application Publication
(10) Pub. No.: US 2021/0330731 A1
(43) Pub. Date: Oct. 28, 2021

(54) TOPICAL SALVE FORMULATIONS FOR DERMAL ALKALOID DELIVERY

(71) Applicant: Robert L Martin, Capitola, CA (US)

(72) Inventor: Robert L Martin, Capitola, CA (US)

(21) Appl. No.: 17/242,129

(22) Filed: Apr. 27, 2021

Related U.S. Application Data
(60) Provisional application No. 63/015,873, filed on Apr. 27, 2020.

(51) Int. Cl. A61K 36/74; A61K 36/24; A61K 36/185; A61K 36/66; A61K 36/28; A61K 36/575; A61K 47/44; A61K 36/899; A61K 31/05; A61K 31/4375; A61K 36/54; A61K 36/23; A61K 36/71; A61K 31/353; A61K 31/20; A61K 45/06; A61K 9/00

(52) U.S. Cl. CPC A61K 36/74; A61K 2236/51; A61K 36/185; A61K 36/66; A61K 36/28; A61K 36/575; A61K 47/44; A61K 36/899; A61K 31/05; A61K 31/4375; A61K 36/54; A61K 36/23; A61K 36/71; A61K 31/353; A61K 31/20; A61K 45/06; A61K 9/0014; A61K 2236/333; A61K 2236/53; A61K 36/24

(57) ABSTRACT

Various formulations of an alkaloid-containing topical analgesic involve extracting alkaloids from herbal material, infusing a carrier oil, and applying secondary ingredients to improve the formulation's therapeutic efficacy. Suitable herbal sources include, but are not limited to, Mitragyna speciosa, Rauwolfia serpentina, Sceletium tortuosum, Harpagophytum procumbens, Picralima natilda, Sceletium tortuosum, Combretum quadrangulare, Corydalis yanhusuo, Lactuca virosa, and/or Magnolia officinalis. Preferably, alcohol extractions of pre-frozen herbal material were vacuum reduced and the resulting alkaloid extraction was infused into an oil and combined with secondary ingredients, such as alkaloid potentiators, dermal absorption enhancers, anti-inflammation agents, brightening agents, neuropathic pain relief agents, and spasm relief agents.

### CLAIM OF PRIORITY

The present application claims benefit of U.S. Provisional Patent Application Ser. No. 63/015,873, titled Topical Salve Formulations Utilizing Alkaloid Extractives of Mitragyna Speciosa, filed Apr. 27, 2020, the entire disclosure of which is hereby expressly incorporated by reference herein.

### FIELD OF TECHNOLOGY

This disclosure relates generally to topical analgesic formulation, and, more particularly, to a pain-relieving analgesic formula utilizing alkaloid extractives from herbal sources such as Mitragyna speciosa.

### BACKGROUND

Topical analgesic formulas are used to temporarily relieve muscle and joint pain locally by applying them directly to skin. Most over the counter (OTC) analgesics are offered in the form of creams, foams, gels, lotions, or ointments and include capsaicin, lidocaine, non-steroidal anti-inflammatory drugs (NSAIDs), counter-irritants, rubefacients, and/or salicylates. Topical analgesics have a negligible system uptake and their therapeutic efficacy is used to treat acute injuries (such as sprains, tendonitis, myalgia, dislocations, and strains) and chronic pains (due to neuropathy or osteoarthritis). However, these analgesics must be reapplied to provide consistent pain relief for those suffering from severe pain. As seen in physical therapy, the reality is that patients with severe pain will consume large amounts of OTC analgesics without experiencing significant pain relief.

Leaves of Mitragyna speciosa (commonly known as ‘kratom’) contain indole alkaloids which have at least partial agonist activity at μ-opioid receptors, which can impart a sedative and sensory-suppressive effect. As such, kratom is often used to manage and de-escalate opioid withdrawal. However, when ingested orally and especially at high relative doses, kratom can impart undesired psychoactive effects, such as nausea, vomiting, dry mouth, incontinence, constipation, aggression, hallucinations, delusions, and thyroid issues. Topical formulations utilizing kratom are rare and those that are available do not absorb through the skin effectively. As such, a topical formulation is needed which does not result in systemic uptake nor impart any psychoactive effects of extracted alkaloids while maximizing skin absorption and potential pain relief without the use of NSAIDs, counter-irritants, and other commonly used but ineffective analgesics.

### SUMMARY

Aspects of various alkaloid-containing topical analgesic formulations are discussed. In one aspect, a method of producing an alkaloid-containing topical analgesic formulation involves producing at least one alkaloid extraction by suspending pre-frozen alkaloid-containing herbal material in a cooled alcohol solution and straining the same. The alkaloid extractions are then vacuum reduced to produced a reduced alkaloid syrup. The reduced alkaloid syrup is then used to infused a carrier oil and produce alkaloid-infused oil. The alkaloid-infused oil is then combined with one or more secondary ingredients, such as alkaloid potentiators, dermal absorption enhancers, anti-inflammation agents, brightening agents, neuropathic pain relief agents, and spasm relief agents. A hardening agent is added and the mixture is emulsified and left to harden.

Other aspects of the topical analgesic formulations and methods of making the same are discussed in further detail below.

### BRIEF DESCRIPTION OF THE DRAWINGS

The embodiments of this invention are illustrated by way of example and not limitation in the figures of the accompanying drawings, in which like references indicate similar elements and in which:

FIG. 1 is a table showing a preferred formulation for a kratom-based topical salve, according to one or more embodiments.

FIG. 2 is a table showing categories and names of ingredients used in most of the embodiments.

FIG. 3 is a process flowchart showing the formulation of an exemplary topical salve formulation for dermal alkaloid delivery.

Other features of the present embodiments will be apparent from the accompanying drawings and from the detailed description that follows.

### DETAILED DESCRIPTION

Formulations of alkaloid-based topical analgesic are expressed which are intended to provide prolonged topical relief for severe pain due to acute or chronic conditions while reducing or preferably eliminating systemic uptake of extracted alkaloids. In general, formulations involve extracting naturally-occurring alkaloids from one or primary herbs comprising kratom (crushed dried leaves of M. speciosa), Rauwolfia serpentina root, Devil's Claw (Harpagophytum procumbens), Akuamma (Picralima natilda), kanna (Sceletium tortuosum), Sakae Naa (Combretum quadrangulare), Corydalis Yanhusuo, Lactuca virosa, and/or Magnolia officinalis and combining them with one or more secondary ingredients oils which are intended for, e.g., changing skin permeability, increasing dermal absorption, adjusting the physical properties of the analgesic formulation, targeting nerve pain, decreasing inflammation and potentiating extracted alkaloids.

The content, concentration, and effects of alkaloids extracted from different herbal sources can vary. Extractives of Lactuca virosa for example, while therapeutically effective, required a higher initial mass of herbal material from which to extract. Known content and/or ranges of alkaloids and other therapeutically effective compounds for various herbal sources include: kratom comprises approximately 1-2.2% mitragynine and 7-o-mitragynine; Akuamma comprises approximately 3.5-4.8% akuammine and alstonine; Corydalis yanhusuo comprises approximately 0.06% on average of several alkaloids, the most potent of which is considered to be tetrahydropalmatine (THP); Rauvolfia comprises reserpine, yohimbine, raubasine, and ajmaline, among other alkaloids; kanna is rich in alkaloids, including but not limited to mesembrine, mesembrenol, mesembrenone, tortusamine; Sakae Naa comprises combretol (an O-methylated flavonol) and 1-O-galloyl-6-O-(4-hydroxy-3,5-dimethoxy)benzoyl-beta-D-glucose, a gallic acid derivative; Magnolia officinalis comprises honokiol and magnolol; and Devil's Claw comprises iridoid glycosides, harpagoside, harpagide, harpagoside-genine, and procumbide.

A botanical extractor may be used to extract alkaloids and/or other active components from the various primary herbs. For example, a simple botanical extractor may involve a double boiler and an immersion blender, which can be used to infuse a batch of oil with plant material and then the plant material can be strained from the oil. However, an alcohol extraction method is preferable because the plant material (which is preferable pre-frozen) can be placed into an alcohol solution (such as ethanol (EtOH)) and strained more promptly and with cleaner results. Such an extraction may be vacuum reduced to reclaim the alcohol from the solution and produce a reduced alkaloid syrup. The reduced alkaloid syrup can be infused into a carrier oil, preferably an oil rich in medium-chain triglycerides (MCTs) such as coconut oil. Additionally, an ultrasonic homogenizer may be used throughout the process. For example, an ultrasonic homogenizer may be used during the extraction to maximize the surface area contact between the herbal material and the alcohol and agitate the solution. In most of the following embodiments, 10 minutes of homogenized alcohol extraction in 90 proof EtOH, straining, a repeated homogenized extraction for 10 minutes, and a vacuum reduction (preferable in a rotary evaporator), was used to yield an optimal concentrate which can be infused in an oil and combined with other secondary ingredients. Ideally, the completed formulation does not stain when applied to skin or if in contact with fabric.

In most embodiments, three-cup batches of oil and wax (e.g.; 2¼ cup oil, ¾ cup wax or other hardening agent) were infused with an extract produced from at least two ounces of plant matter processed using the above method. In a preferred embodiment, an extract produced from around 4.5 to 6 ounces of herbal material yielded the best results as far as optimal pain relief for the amount of product used. More than 6 oz in a 3-cup infusion had no increased effect, i.e., resulting formulations did not relieve pain any better.

In one embodiment, a formulation for a kratom alkaloid-based topical product involves extracting alkaloids from kratom powder into a food-grade oil (such as coconut oil) at 160° F. The powder may be combined with a lecithin (such as a sunflower lecithin) to aid in extraction. This base formulation has a noticeable therapeutic effect but is considerably improved by the addition of other materials. The extraction was also performed at 180° F. and 220° F. with no noticeable difference in potency.

In a further embodiment, a formulation involves extracting alkaloids from kratom powder into a food-grade oil at 160° F. for two hours and adding one or more carrier oils. The carrier oils comprise black pepper oil, turmeric oil, peppermint oil and lemon oil. A hardening agent, such as beeswax, may also be added. Piperine (a black pepper alkaloid), curcumin (a metabolite of turmeric), peppermint oil, and lemon oil are intended as dermal carriers and absorption enhancers. Piperine is known to increase curcumin bioavailability. This carrier oil formulation had a significantly stronger therapeutic potency compared to the base formulation.

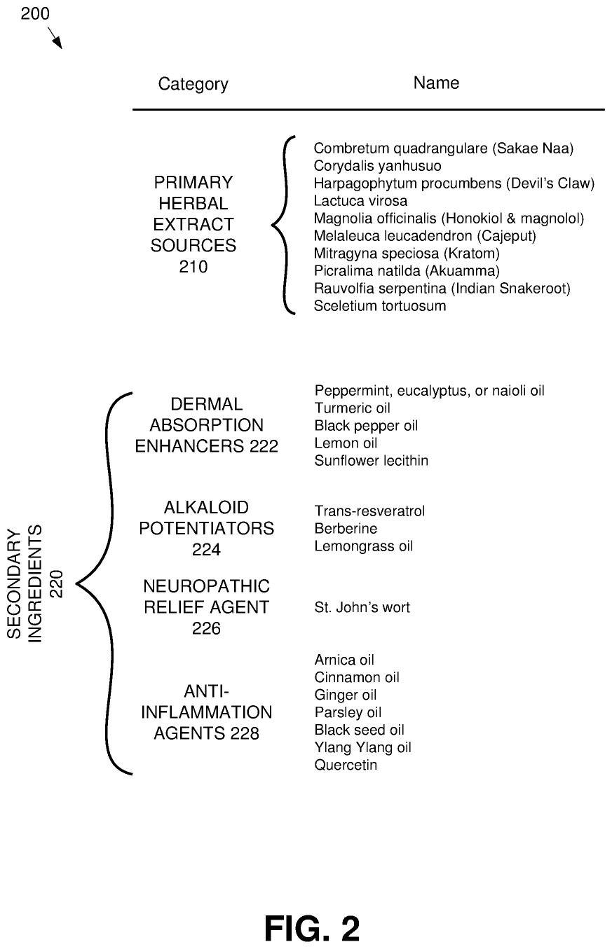

In a further embodiment, a severe pain relief formulation involves extracting alkaloids from kratom powder into a food-grade oil at 160° F. for two hours and adding one or more carrier oils. The carrier oils comprise black pepper oil, turmeric oil, peppermint oil (alternately, eucalyptus or naioli oils will suffice since they contain similar compounds), lemon oil, magnesium oil, St John's Wort oil, arnica oil, parsley oil, cinnamon oil, lemongrass oil, ginger oil, arnica oil (comprises helenalin, fatty acids (inolenic, palmitic, linoleic, and myristic) thymol, ethers of thymol, thymohydroquinone dimethyl ether, and phlorol isobutyrate (2%)), Ylang Ylang (Cananga odorata) oil (comprises linalool, caryophyllene and germacrene), and/or Black Seed oil (comprises anethole, p-cymene, limonene, carvone and thymoquinone). The addition of magnesium, St John's Wort, arnica, and ginger oils served to provide added relief for severe neuropathic pain and muscle spasms. FIG. 2 provides a table 200 listing primary herbal extract sources 210 and secondary ingredients 220, including but not limited to dermal absorption enhancers 222, alkaloid potentiators 224, neuropathic relief agents 226, and anti-inflammation agents 228.

In yet another embodiment, a severe pain and inflammation relief formulation involves extracting alkaloids from kratom powder into a food-grade oil at 160° F. for two hours and adding one or more carrier oils. The carrier oils comprise black pepper oil, turmeric oil, peppermint oil, lemon oil, magnesium oil, St. John's Wort oil, arnica oil, and ginger oil. The kratom powder may be a combination of concentrated kratom extract and kratom powder. This formulation has proved to have a positive therapeutic effect for patients with scoliosis, migraines, chronic joint pain, inflammation, chronic fatigue syndrome, fibromyalgia, cervical disc injury, and nerve compression.

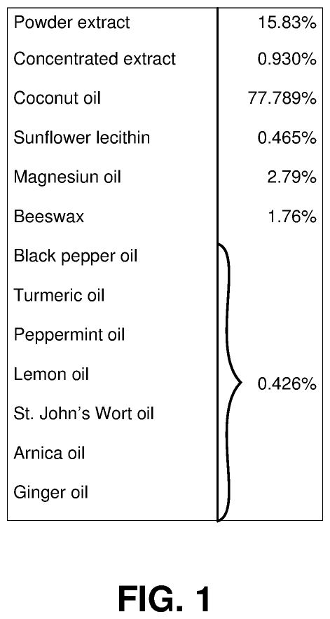

In a preferred embodiment for a kratom concentrate-based topical analgesic as shown in FIG. 1, a severe pain and inflammation relief formulation involves extracting alkaloids from a combination of kratom powder (15.83%) and concentrated kratom extract (0.930%) into extra-virgin, cold pressed coconut oil (77.79%) at 160° F. for two hours with sunflower lecithin (0.465%) as an emulsifier. Soy lecithin may be avoided to prevent any negative effects due its estrogenic properties. Additionally, a plurality of carrier oils (0.428%) may be added and may comprise black pepper oil, turmeric oil, peppermint oil, lemon oil, St. John's Wort oil, arnica oil, and ginger oil. Although an equal ratio may be used, an additional proportion of black pepper oil and turmeric oil is preferred in order to emphasize their multiplicative properties as far as alkaloid absorption is concerned. Magnesium oil (2.79%) is added as a muscle and nerve relaxant and beeswax (1.76%) is also added to harden the formulation. Instead of combining powder and extract, a commensurate amount of extract may be used alone. Extract potency may vary based on the particular strain of M. Speciosa used. For example, when utilizing ‘Kraken Gold Kratom Extract’ brand extract, 5 gm is approximately equivalent to 1.5 oz of kratom powder (approximately 8.5 times more potent).

Alkaloid potentiators may additionally be used to improve the efficacy of alkaloid delivery by augmenting and/or prolonging the pain-relieving effects of the formulation. Trans-resveratrol, a polyphenol found naturally in plants responding to injury or attack by pathogens, was found to increase the effect of a formulation by approximately two hours or longer. Berberine also produced a similar effect when added in small amounts, but stained the formulation bright yellow. Another alkaloid potentiator is lemongrass oil. Other secondary ingredients that produced notable effects include quercetin (decreased pain and inflammation), magnesium stearate (relieved muscle spasms), copper salicylate (minor improvement to pain relief), and zinc oxide (brightens formulas made with darker extracts such as kratom and Akuamma).

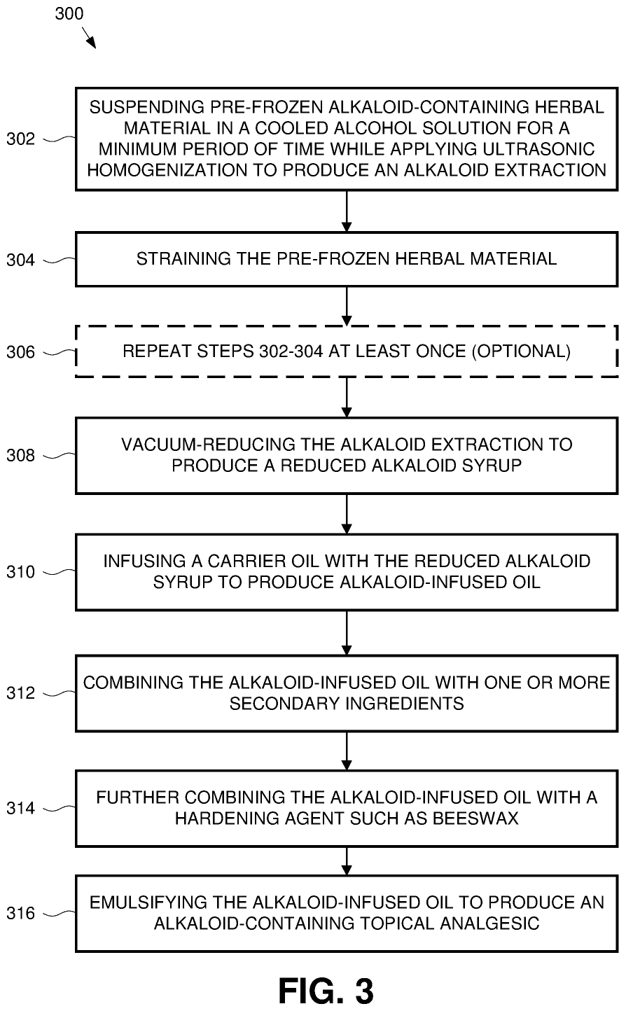

Referring to FIG. 3, a method of producing an exemplary alkaloid-containing topical analgesic formulation is shown. Firstly, step 302 involves suspending pre-frozen alkaloid-containing herbal material in a cooled alcohol solution for a minimum period of time while applying ultrasonic homogenization to produce an alkaloid extraction. For example, 4-6 ounces of herbal material may be mixed with approximately 5 cups of alcohol (EtOH) in a stainless-steel container. The container may be placed in a larger container containing ice water to maintain a cold temperature. Citric acid may also be added to the extraction to improve alkaloid solubility (approximately ½ tsp). The minimum period of time for suspension while applying ultrasonic homogenization may be approximately 10 minutes at 90% pulse intensity. In a step 304, the pre-frozen herbal material is strained from the alcohol solution. Straining may occur multiple times, preferably three, wherein each straining utilizes a filter of decreasing filter size. For example, the herbal material may be strained first with a 400-micron filter, then strained again with a 300-micron filter, and again with a 200-micron filter. The extraction and straining steps 302-304 may be repeated in an optional step 306 as required to create multiple alkaloid extractions.

In a step 308, the alkaloid extraction(s) is/are vacuum-reduced to produce a reduced alkaloid syrup. The alcohol may be reclaimed for reuse. In a step 310, a carrier oil (such as coconut oil) is infused with the alkaloid syrup. For example, coconut may be used (approximately 4-5 cups of oil for the extractions described above). Sunflower lecithin may be added to aid in emulsifying the alkaloid syrup and oil mixture. A proportion of 1 tsp sunflower lecithin for 1 cup of oil is preferable. A step 312 involves combining the alkaloid-infused oil with one or more secondary ingredients (see FIG. 2) and homogenizing the mixture again. In step 314, melted beeswax (1 part beeswax per 6 parts oil) is added to the alkaloid-infused oil. In a step 316, the alkaloid-infused oil is emulsified again and left to harden (approximately 24 hours). After hardening, a layer of sediment may settle at the bottom below a layer of saturated oils. The saturated oils above the sediment layer may be withdrawn, remelted, and poured into individual, small containers and used as an alkaloid-containing topical analgesic.

Although the present embodiments have been described with reference to specific example embodiments, it will be evident that various modifications and changes may be made to these embodiments without departing from the broader spirit and scope of the various embodiments. It is to be understood that the specific order or hierarchy of steps in the methods disclosed is an illustration of exemplary processes. Based upon therapeutic preferences, it is understood that the specific order or hierarchy of steps in the methods may be rearranged. The accompanying method claims present elements of the various steps in a sample order, and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. A method of formulating a topical analgesic, comprising:
producing one or more alkaloid extractions, comprising:
suspending pre-frozen alkaloid-containing herbal material in a cooled alcohol solution for a minimum period of time while applying ultrasonic homogenization to produce an alkaloid extraction;
straining the pre-frozen alkaloid-containing herbal material;
vacuum-reducing the alkaloid extraction(s) to produce a reduced alkaloid syrup;
infusing a carrier oil with the reduced alkaloid syrup to produce alkaloid-infused oil;
combining the alkaloid-infused oil with one or more secondary ingredients selected from the group consisting of: alkaloid potentiators, dermal absorption enhancers, anti-inflammation agents, brightening agents, neuropathic pain relief agents, and spasm relief agents;
hardening the alkaloid-infused oil with a hardening agent; and
emulsifying the alkaloid-infused oil to produce an alkaloid-containing topical analgesic.

2. The method of claim 1, wherein the herbal material comprises herbal matter selected from the group consisting of: Mitragyna speciosa, Rauwolfia serpentina, Sceletium tortuosum, Harpagophytum procumbens, Picralima natilda, Sceletium tortuosum, Combretum quadrangulare, Corydalis yanhusuo, Lactuca virosa, and/or Magnolia officinalis.

3. The method of claim 1, wherein the dermal absorption enhancers comprise turmeric oil, black pepper oil, peppermint oil, eucalyptus oil, naioli oil, lemon oil, and sunflower lecithin.

4. The method of claim 1, wherein the alkaloid potentiatiors comprise trans-resveratrol, berberine, and lemongrass oil.

5. The method of claim 1, wherein the neuropathic pain relief agents comprise St. John's wort.

6. The method of claim 1, wherein the anti-inflammation agents comprise arnica oil, cinnamon oil, ginger oil, parsley oil, black seed oil, Ylang Ylang essential oil, and quercetin.

7. The method of claim 1, wherein the spasm relief agents comprise magnesium stearate.

8. The method of claim 1, wherein suspending pre-frozen herbal matter in a cooled alcohol solution additionally comprises placing a container containing the pre-frozen herbal matter and the cooled alcohol solution in an ice water bath.

9. The method of claim 1, wherein suspending pre-frozen herbal matter in a cooled alcohol solution additionally comprises adding citric acid to improve alkaloid extraction.

10. The method of claim 1, wherein straining the pre-frozen herbal matter involves straining at least once with a plurality of filters of decreasing micron size, from approximately 400 microns to approximately 200 microns.

11. A concentrated topical analgesic formulation, comprising:
an alkaloid extraction from pre-frozen alkaloid-containing herbal matter;
wherein the alkaloid extraction is vacuum reduced post extraction;
a carrier oil capable of solubilizing the alkaloid extraction;
a firming agent; and
one or more secondary ingredients selected from the group consisting of: alkaloid potentiators, dermal absorption enhancers, anti-inflammation agents, brightening agents, neuropathic pain relief agents, and spasm relief agents.

12. The formulation of claim 11, wherein the herbal matter comprises herbal matter selected from the group consisting of: Mitragyna speciosa, Rauwolfia serpentina, Sceletium tortuosum, Harpagophytum procumbens, Picralima natilda, Sceletium tortuosum, Combretum quadrangulare, Corydalis yanhusuo, Lactuca virosa, and/or Magnolia officinalis.

13. The formulation of claim 11, wherein the dermal absorption enhancers comprise turmeric oil, black pepper oil, peppermint oil, eucalyptus oil, naioli oil, lemon oil, and sunflower lecithin.

14. The formulation of claim 11, wherein the alkaloid potentiatiors comprise trans-resveratrol, berberine, and lemongrass oil.

15. The formulation of claim 11, wherein the neuropathic pain relief agents comprise St. John's wort.

16. The formulation of claim 11, wherein the anti-inflammation agents comprise arnica oil, cinnamon oil, ginger oil, parsley oil, black seed oil, Ylang Ylang essential oil, and quercetin.

17. The formulation of claim 1, wherein the spasm relief agents comprise magnesium stearate.
